(19) United States
(12) Patent Application Publication
(10) Pub. No.: US 2010/0317957 A1
(43) Pub. Date: Dec. 16, 2010

(54) THREE-DIMENSIONAL WEARABLE ELECTRODE SET

(75) Inventors: James LEE, Taipei (TW); Hong Hsu HUANG, TAIPEI (TW)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(73) Assignees: TEX-RAY INDUSTRIAL CO., LTD., TAIPEI (TW); KING'S METAL FIBER TECHNOLOGIES CO., LTD., TAIPEI (TW)

(21) Appl. No.: 12/485,271

(22) Filed: Jun. 16, 2009

(51) Int. Cl. A61B 5/0408; A61N 1/04; A61B 5/0492

(52) U.S. Cl. 600/388; 607/149

(57) ABSTRACT

A three-dimensional (3D) wearable electrode set for a human body is provided. The 3D wearable electrode set comprises an integrative first ring electrode and an integrative second ring electrode. The integrative first ring electrode has a conductive layer and a ring basis, and the integrative second ring electrode also has a conductive layer and a ring basis. The conductive layers of the integrative first and second ring electrodes are adopted to cover around the first and second portions of the human body, respectively. The conductive layers are formed with conductive material. The ring bases are formed with insulating fabric.

### CROSS-REFERENCES TO RELATED APPLICATIONS

Not applicable.

### BACKGROUND OF THE INVENTION

1. Field of the Invention

The present invention provides a three-dimensional (3D) wearable electrode set. More specifically, the present invention provides a three-dimensional wearable electrode set without electric adhesive patches.

2. Descriptions of the Related Art

In medical domain, it is important to keep tracking patients' physiological statuses, such as myoelectricity status or cardiac reflex status. In general, the myoelectricity status is sensed according to electromyography (EMG) signals, and the cardiac reflex status is sensed according to electrocardiograph (ECG) signals.

Typically, the EMG signals or ECG signals are able to be measured via a plurality of electric adhesive patches adhered on a human body. More specifically, these electric adhesive patches are adhered on different portions of the human body to sense the EMG signals or ECG signals, and are electrically connected to a monitor to analyze the signals for displaying.

Furthermore, to be more convenient, the industry integrates these electric adhesive patches with a garment, such as a T-shirt. In other words, these electric adhesive patches are adhered on the garment and in close contact with the skin of the human body, so that for sportsmen, they can be notified whether the exercises is effective or not by checking the ECG signals while they are taking exercises.

However, the electric adhesive patches will lose their adhesive and cause folder over in such a manner that the physiological signals can not be measured or are not reliable. In fact, there is a high possibility for sportsmen that the electric adhesive patches fall off from the garment during their exercises.

In view of this, it is important to provide wearable electrodes that are convenient for use and are not requiring the electric adhesive patches.

### SUMMARY OF THE INVENTION

The primary objective of this invention is to provide a 3D wearable electrode set for a human body, which comprises an integrative first ring electrode and an integrative second ring electrode. The integrative first ring electrode which has a first conductive layer and a first ring basis is adopted to cover around a first portion of the human body. The integrative second ring electrode which has a second conductive layer and a second ring basis is adopted to cover around a second portion of the human body. The first and second conductive layers are formed with conductive material, and the first and second ring bases are formed with insulating fabric. The first conductive layer is attached to the first ring basis, and the second conductive layer is attached to the second ring basis. The first conductive layer is electrically connected to a first terminal of a processor via a first conductive thread, and the second conductive layer is electrically connected to a second terminal of the processor via a second conductive thread.

Accordingly, the 3D wearable electrode set comprising the integrative first and second ring electrodes do not require electric adhesive patches to sense the myoelectricity status or the cardiac reflex status of a human body, and are able to cover around different portions of the human body which may give a better and more accurate results than the electric adhesive patches of the prior art.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a schematic view illustrating a preferred embodiment of the present invention;

FIGS. 2A-2C are schematic views illustrating the integrative first and second ring electrodes of the preferred embodiment;

FIG. 3A is a schematic view illustrating conductive fabric of the preferred embodiment;

FIG. 3B is a schematic view illustrating insulating fabric of the preferred embodiment; and

FIGS. 4A-4B are schematic views illustrating the integrative first and second ring electrodes of another preferred embodiment.

### DESCRIPTION OF THE PREFERRED EMBODIMENT

In the following description, this invention will be explained with reference to embodiments thereof. However, these embodiments are not intended to limit this invention to any specific environment, applications or implementations described in these embodiments. Therefore, description of these embodiments is only for purposes of illustration rather than to limit the present invention. It should be appreciated that in the following embodiments and the attached drawings, the elements not related directly to this invention are omitted from depiction.

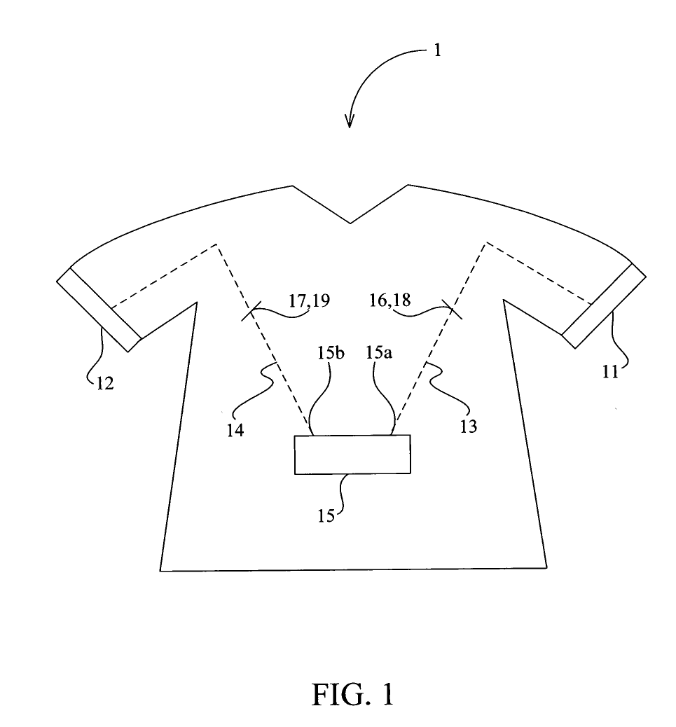

FIG. 1 is a schematic view illustrating a preferred embodiment of a garment 1 incorporated with a 3D wearable electrode set of the present invention. The 3D wearable electrode set comprises an integrative first ring electrode 11 and an integrative second ring electrode 12. The integrative first ring electrode 11 and the integrative second ring electrode 12 may be sewed on the two sleeves of the garment 1. In other embodiments, the integrative first ring electrode 11 and the integrative second ring electrode 12 may form with the garment 1 as a piece rather than an addition of the garment 1. The integrative first ring electrode 11 is electrically connected to a first terminal 15a of a processor 15 via a first conductive thread 13, and the integrative second ring electrode 12 is electrically connected to a second terminal 15b of the processor 15 via a second conductive thread 14. In this preferred embodiment, the first conductive thread 13 and the second conductive thread 14 are yarns blended with the conductive fibers and the insulating fibers. It should be noted that the first conductive thread 13 and the second conductive thread 14 are made by pure conductive fibers, conductive wires, or conductive inks, the present invention has no intention to limit the material of the first conductive thread 13 and the second conductive thread 14 as long as their material are conductive.

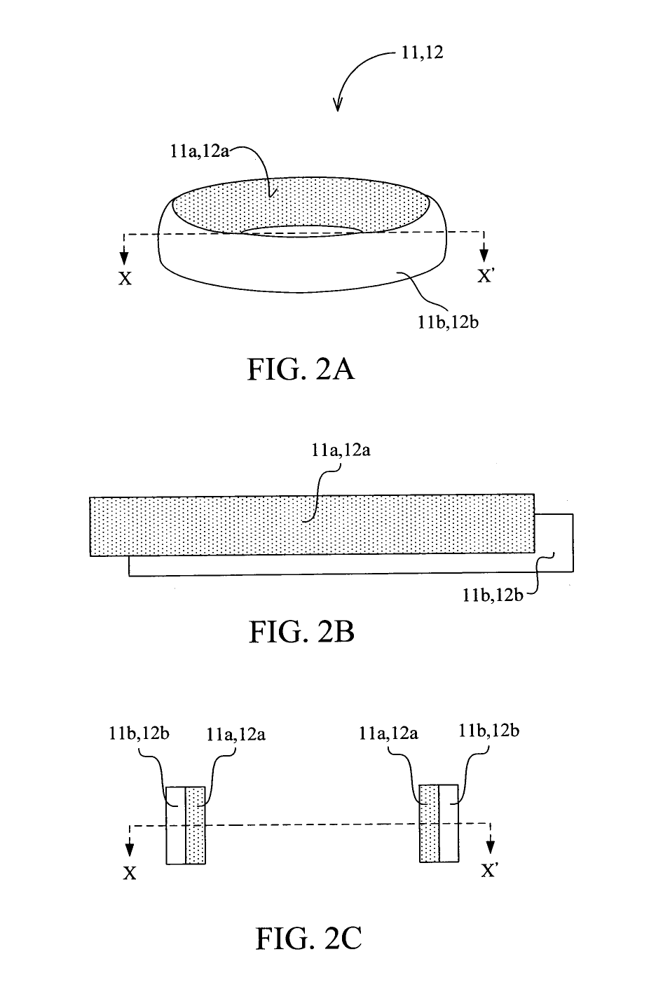

FIGS. 2A-2C are schematic views of the integrative first ring electrode 11 or the integrative second ring electrode 12 in accordance with the garment 1 of the preferred embodiment. More specifically, FIG. 2A is a schematic views enlarging the integrative first ring electrode 11 or the integrative second ring electrode 12, FIG. 2B is an exploded view of different layers of the integrative first ring electrode 11 or the integrative second ring electrode 12, and FIG. 2C is a cross sectional view of the integrative first ring electrode 11 or the integrative second ring electrode 12 in accordance with X-X′.

The integrative first ring electrode 11 has a first conductive layer 11a and a first ring basis 11b, wherein the first conductive layer 11a is attached to the first ring basis 11b, and the first conductive layer 11a is woven with conductive fibers and insulating fibers while the first ring basis 11b is formed with insulating fabric.

Similarly, the integrative second ring electrode 12 has the same structure as the integrative first ring electrode 11. In other words, the integrative second ring electrode 12 has a second conductive layer 12a and a second ring basis 12b, wherein the second conductive layer 12a is attached to the second ring basis 12b, and the second conductive layer 12a is woven with conductive fibers and insulating fibers while the second ring basis 12b is formed with insulating fabric.

In this preferred embodiment, the first conductive layer 11a has a ring structure, and a surface area of that is equal to a surface area of the first ring basis 11b. The second conductive layer 12a also has the ring structure, and a surface area of that is equal to a surface area of the second ring basis 12b.

In FIG. 2C, the first conductive layer 11a and the first ring basis 11b on the right side of FIG. 2C are refer to a right portion of the integrative first ring electrode 11 in FIG. 2A. Similarly, the second conductive layer 12a and the second ring basis 12b on the right side of FIG. 2C are refer to a right portion of the integrative second ring electrode 12 in FIG. 2A.

On the other hand, the first conductive layer 11a and the first ring basis 11b on the left side of FIG. 2C are refer to a left portion of the integrative second ring electrode 12 in FIG. 2A, and the second conductive layer 12a and the second ring basis 12b on the left side of FIG. 2C are refer to a left portion of the integrative second ring electrode 12 in FIG. 2A.

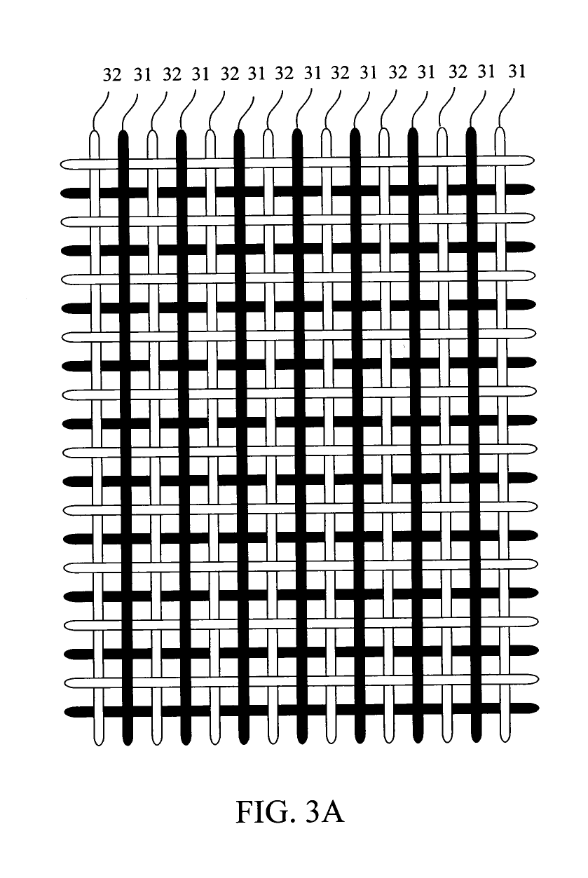

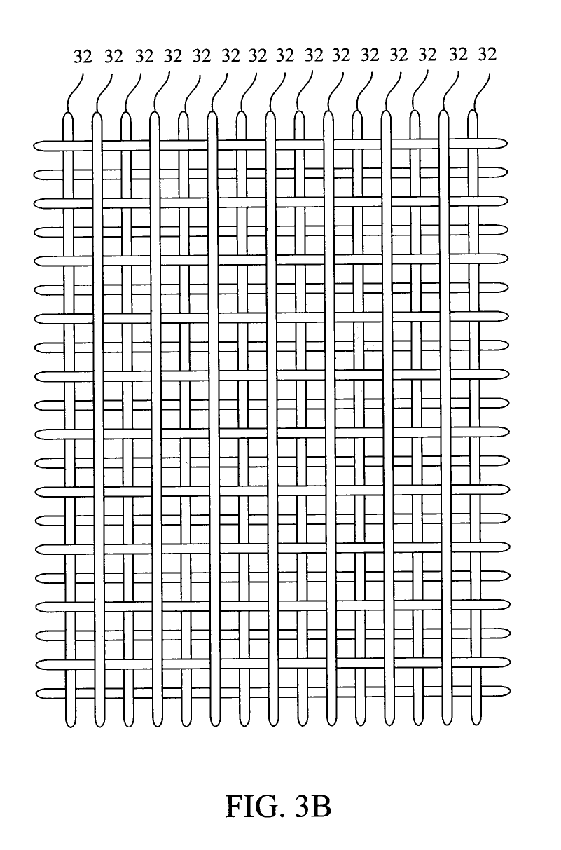

Furthermore, the first conductive layer 11a and the second conductive layer 12a are woven with conductive fibers 31 and insulating fibers 32 in such a manner as shown in FIG. 3A, wherein material of the conductive fibers 31 is metal fibers with electric conductively, such as stainless steel. In other embodiments, the first conductive layer 11a and the second conductive layer 12a may be formed with fabric applied conductive ink or conductive paint.

On the other hand, the first ring basis 11b and the second ring basis 12b are woven with a plurality of insulating fibers 32 in such a manner as shown in FIG. 3B, wherein material of the insulating fibers 31 is conventional fibers without electric conductively, such as cotton fibers. It should be noted that the first conductive layer 11a and the second conductive layer 12a can be made by conductive inks, the present invention has no intention to limit the material of the first conductive layer 11a and the second conductive layer 12a as long as their material are conductive.

Please refer to FIGS. 1˜2C, the 3D wearable electrode set is for a human body. The integrative first ring electrode 11 and the integrative second ring electrode 12 are made by elastic material, so that the integrative first ring electrode 11 and the integrative second ring electrode 12 are able to slight tight round a first portion (e.g. the right elbow) and a second portion (e.g. a left elbow) of the human body respectively.

Further speaking, the first conductive layer 11a and the second conductive layer 12a contact with the skin of the first potion and the second portion of the human body and are electrically connected to the first terminal 15a and the second terminal 15b of the processor 15 via the first conductive thread 13 and the second conductive thread 14 respectively. Therefore, the human body may receive some electric stimulation from the processor 15 via the first conductive layer 11a and the second conductive layer 12a.

For example, the 3D wearable electrode set may be for diathermy, such as transcutaneous electrical nerve stimulation (TENS). The processor 15 may generate a first simulation current 16 and a second simulation current 17. The first simulation current 16 is transmitted to the first conductive layer 11a of the integrative first ring electrode 11 via the first conductive thread 13, and the second simulation current 17 is transmitted to the second conductive layer 12a of the integrative second ring electrode 12 via the second conductive thread 14. According to the first simulation current 16 and the second simulation current 17 from the processor 15, the diathermy can be easily achieved by the processor 15, the integrative first ring electrode 11 and the integrative second ring electrode 12.

On the other hand, if the 3D wearable electrode set is for monitoring the myoelectricity status or the cardiac reflex status of the human body, the first conductive layer 11a may receive a first electrical impulse 18 (e.g. one of the EMG signals or one of the ECG signals) generated from the first portion of the human body, and then the first electrical impulse 18 is transmitted to the first terminal 15a of the processor 15 via the first conductive thread 13. Similarly, the second conductive layer 12a may receive a second electrical impulse 19 (e.g. another EMG signal or another ECG signal) generated from the second portion of the human body, and then the second electrical impulse 19 is transmitted to the second terminal 15b of the processor 15 via the second conductive thread 14.

After receiving the first electrical impulse 18 and the second electrical impulse 19, the processor 15 analyzes the impulses 18, 19 to retrieve the electromyogram or the electrocardiogram of the human body. According to the first electrical impulse 18 and the second electrical impulse 19 from the integrative first ring electrode 11 and the integrative second ring electrode 12, monitoring of the myoelectricity status or the cardiac reflex status of the human body can be easily achieved by the processor 15, the integrative first ring electrode 11 and the integrative second ring electrode 12. In other embodiments, the integrative first ring electrode 11 and the integrative second ring electrode 12 may additionally sense heart beat pulse.

It should be noted that even though the 3D wearable electrode set are incorporated with garment 1 in this preferred embodiment as shown in FIG. 1, the 3D wearable electrode set may incorporated with a long sleeve sweater, or a sport pant for allow to cover around arms, feet, thigh, belly or even neck of the body. Furthermore, to obtain a better and more accurate the myoelectricity status or the cardiac reflex status, the 3D wearable electrode set must comprises at lest two integrative ring electrodes to cover around different portions of the human body, people skilled in this art may rapidly add more integrative ring electrodes since the structures of integrative ring electrodes are basically the same. For the cardiac reflex status, it is even desired to place the integrative ring electrodes across the heart, for example, the integrative ring electrodes are placed at right elbow and left ankle.

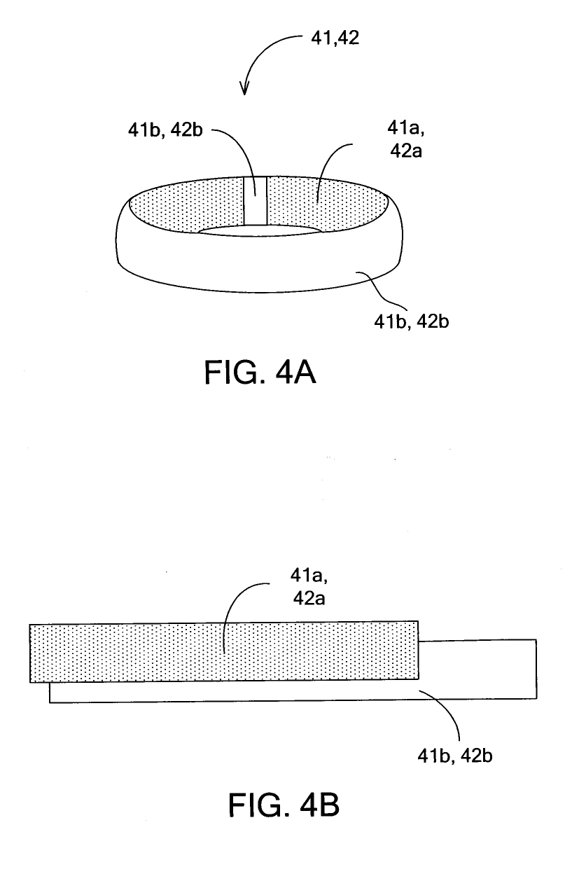

FIGS. 4A-4B are schematic views of an integrative first ring electrode 41 or an integrative second ring electrode 42 of another preferred embodiment. More specifically, FIG. 4A is a schematic view enlarging the integrative first ring electrode 41 or the integrative second ring electrode 42, and FIG. 4B is an exploded view of different layers of the integrative first ring electrode 41 or the integrative second ring electrode 42. The integrative first ring electrode 41 or the integrative second ring electrode 42 may combined with the garment 1 of the prior preferred embodiment, the details are described as above and therefore will not be mentioned here.

The integrative first ring electrode 41 has a first conductive layer 41a and a first ring basis 41b, wherein a surface area of the first conductive layer 41a is less than a surface area of the first ring basis 41b, and the first conductive layer 41a is attached to a part of the first ring basis 41b. Preferably, the surface area of the first conductive layer 41a holds a ratio between 20% and 80% of the surface area of the first ring basis 41b. The first conductive layer 41a is woven with conductive fibers and insulating fibers while the first ring basis 41b is formed with insulating fabric.

Similarly, the integrative second ring electrode 42 has the same structure as the integrative first ring electrode 41. In other words, the integrative second ring electrode 42 has a second conductive layer 42a and a second ring basis 42b. A surface area of the second conductive layer 42a is less than a surface area of the second ring basis 42b, and the second conductive layer 42a is attached to a part of the second ring basis 42b. Preferably, a surface area of the second conductive layer 42a holds a ratio between 20% and 80% of a surface area of the second ring basis 42b. The second conductive layer 42a is woven with conductive fibers and insulating fibers while the second ring basis 42b is formed with insulating fabric.

Because the surface area of the first conductive layer 41a/the second conductive layer 42a is less than the surface area of the first ring basis 41b/the second ring basis 42b, so that a conductive area of the integrative first ring electrode 41 or the integrative second ring electrode 42 are discontinuous as shown in FIG. 4A, which is different from integrative first ring electrode 11 and the integrative second ring electrode 12 as shown in FIG. 2A.

The first conductive layer 41a and the second conductive layer 42a are woven with conductive fibers 31 and insulating fibers 32 in such a manner as shown in FIG. 3A, wherein material of the conductive fibers 31 is metal fibers with electric conductively, such as stainless steel. The first ring basis 41b and the second ring basis 42b are woven with a plurality of insulating fibers 32 in such a manner as shown in FIG. 3B, wherein material of the insulating fibers 31 is conventional fibers without electric conductively, such as cotton fibers.

Similarly, the first conductive layer 41a and the second conductive layer 42a may contact with the skin of the first potion and the second portion of the human body and are electrically connected to the first terminal (not shown) and the second terminal (not shown) of the processor (not shown) via the first conductive thread (not shown) and the second conductive thread (not shown) respectively. Therefore, the human body may receive some electric stimulation from the processor via the first conductive layers and the second conductive layers. The details for TENS and monitoring the myoelectricity status or the cardiac reflex status of the human body applications are already described in the previous preferred embodiment and therefore will not be mentioned here.

Accordingly, the 3D wearable electrode set which comprises the integrative first ring electrode and the integrative first ring electrode does not require electric adhesive patches, and is able to cover around different portions of the human body which may give a better and more accurate results than the electric adhesive patches of the prior art. Hence, the problem of the prior art is overcome.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A three-dimensional (3D) wearable electrode set for a human body, comprising:
an integrative first ring electrode, having a first conductive layer and a first ring basis, being adopted to cover around a first portion of the human body, wherein the first conductive layer is formed with conductive material, the first ring basis is formed with insulating fabric, and the first conductive layer is attached to the first ring basis; and
an integrative second ring electrode, having a second conductive layer and a second ring basis, being adopted to cover around a second portion of the human body, wherein the second conductive layer is formed with the conductive material, the second ring basis is formed with the insulating fabric, and the second conductive layer is attached to the second ring basis;
wherein the first conductive layer is electrically connected to a first terminal of a processor via a first conductive thread, the second conductive layer is electrically connected to a second terminal of the processor via a second conductive thread.

2. The 3D wearable electrode set of claim 1, wherein the first conductive thread and second conductive thread are yarns blended with the conductive fibers and the insulating fibers.

3. The 3D wearable electrode set of claim 1, wherein the processor generates a first stimulation current and a second stimulation current, the first stimulation current is transmitted to the integrative first ring electrode via the first conductive thread and the first terminal of the processor, and the second stimulation current is transmitted to the integrative second ring electrode via the second conductive thread and the second terminal of the processor.

4. The 3D wearable electrode set of claim 3, wherein the first stimulation current and the second stimulation current are used for transcutaneous electrical nerve stimulation (TENS).

5. The 3D wearable electrode set of claim 1, wherein the first conductive layer receives a first electrical impulse generated from the first portion of the human body and transmits the first electrical impulse to the first terminal of the processor via the first conductive thread.

6. The 3D wearable electrode set of claim 5, wherein the second conductive layer receives a second electrical impulse generated from the second portion of the human body and transmits the second electrical impulse to the second terminal of the processor via the second conductive thread.

7. The 3D wearable electrode set of claim 6, wherein the first electrical impulse and the second electrical impulse forms an electromyography (EMG) signal.

8. The 3D wearable electrode set of claim 6, wherein the first electrical impulse and the second electrical impulse forms an electrocardiography (ECG) signal.

9. The 3D wearable electrode set of claim 1, wherein the conductive material is a fabric woven with conductive fibers and insulating fibers.

10. The 3D wearable electrode set of claim 1, wherein the integrative first ring electrode and the integrative second ring electrode are made by elastic material.

11. The 3D wearable electrode set of claim 1, wherein a surface area of the first conductive layer is less than a surface area of the first ring basis, and a surface area of the second conductive layer is less than a surface area of the second ring basis.

12. The 3D wearable electrode set of claim 1, wherein the first conductive layer substantially has a ring structure, a surface area of the first conductive layer is equal to a surface area of the first ring basis, the second conductive layer substantially has the ring structure, and a surface area of the second conductive layer is equal to a surface area of the second ring basis.
